(19) United States
(12) Patent Application Publication
(10) Pub. No.: US 2015/0072014 A1
(43) Pub. Date: Mar. 12, 2015

(54) Pharmaceutical Compositions of Sodium Picosulfate, Magnesium Oxide and Citric Acid

(71) Applicant: CADILA HEALTHCARE LIMITED, Ahmedabad (IN)

(72) Inventors: Kannan Muthaiyyan ESSAKIMUTHU, Ahmedabad (IN); Jitendra Rameshchandra PATEL, Ahmedabad (IN); Paritosh Kunwar SINGH, Ahmedabad (IN)

(73) Assignee: CADILA HEALTHCARE LIMITED, Ahmedabad (IN)

(21) Appl. No.: 14/482,678

(22) Filed: Sep. 10, 2014

(30) Foreign Application Priority Data
Sep. 10, 2013 (IN) 2911/MUM/2013

(51) Int. Cl. A61K 31/4402; A61K 47/26; A61K 9/14; A61K 47/02; A61K 47/12

(52) U.S. Cl. CPC A61K 31/4402; A61K 47/02; A61K 47/12; A61K 9/14; A61K 9/145; A61K 47/26
USPC 424/490; 514/277; 424/489

(57) ABSTRACT

The present invention relates to pharmaceutical compositions of sodium picosulfate, magnesium oxide and citric acid. In particular, the invention relates to pharmaceutical compositions comprise granules having a layer of sodium picosulfate on neutral carrier/s. The invention also relates to processes for the preparation of such compositions and use thereof for bowel cleansing.

### FIELD OF THE INVENTION

The present invention relates to pharmaceutical compositions of sodium picosulfate, magnesium oxide and citric acid. In particular, the invention relates to pharmaceutical compositions comprising granules having a layer of sodium picosulfate on a neutral carrier. The invention also relates to processes for the preparation of such compositions and use thereof for bowel cleansing.

### BACKGROUND OF THE INVENTION

A pharmaceutical product used for clearance of the bowel prior to X-ray examination, endoscopy or surgery, is presently sold under the trade mark name of PICOLAX™. The pharmaceutical product is a white powder which is made up as a solution (in water) for administration. The pharmaceutical product includes sodium picosulfate, a stimulant laxative; anhydrous citric acid and magnesium oxide, which together in solution form magnesium citrate, an osmotic laxative with a powerful cathartic effect.

The known process for making PICOLAXT™ may include the following steps. Granules of magnesium oxide and citric acid are produced by mixing the two reagents together—this is known as the “primary mix”. In another stage, potassium bicarbonate, sodium picosulfate and water are mixed or blended to produce a wet “pre-mix”, which is then dried. In a further stage, the flavor ingredients, orange flavor and sodium saccharin, are blended with the pre-mix and primary mix. The known process has several associated problems.

The mixing processes may result in inhomogeneity problems in the final and intermediate products. In one aspect, the terms “inhomogeneity” and “lack of homogeneity” as used in this application refer to the lack of uniformity of content of the active substance—sodium picosulfate in the final product. It also refers to the lack of homogeneity in the physical and morphological properties, such as the particle size (diameter) or particle size range or distribution, of the intermediate products and/or the final product granules. Intermediate product granules are, for example the primary mix granules or the pre-mix granules.

US Publication no. 2011/0104285 discloses granular composition comprising magnesium oxide coated on citric acid.

US Publication no. 2012/0015036 discloses granular composition comprising sodium picosulfate spray coated on the potassium bicarbonate core.

Hence, there still remains a need for alternate pharmaceutical compositions comprising sodium picosulfate, magnesium oxide and citric acid in order to achieve homogeneity in the physical and morphological properties, such as the particle size (diameter) or particle size range or distribution, of the intermediate products and/or the final product granules.

### SUMMARY OF THE INVENTION

In one general aspect there is provided a pharmaceutical composition comprising sodium picosulfate, magnesium oxide, citric acid and one or more pharmaceutically acceptable excipients.

In another general aspect there is provided a pharmaceutical composition comprising sodium picosulfate, magnesium oxide, citric acid, an alkaline agent and one or more pharmaceutically acceptable excipients.

In another general aspect there is provided a pharmaceutical composition comprising sodium picosulfate, magnesium oxide, citric acid and one or more pharmaceutically acceptable excipients, wherein sodium picosulfate is coated on a neutral carrier.

In another general aspect there is provided a pharmaceutical composition comprising:
a) granules of sodium picosulfate and a neutral carrier, and
b) a mixture comprising citric acid, magnesium oxide, an alkaline agent and one or more pharmaceutically acceptable excipients.

In another general aspect there is provided a pharmaceutical composition comprising: granules of sodium picosulfate and lactose; and a mixture comprising citric acid, magnesium oxide, potassium bicarbonate and one or more pharmaceutically acceptable excipients, where in sodium picosulfate is coated onto lactose.

In another general aspect there is provided a pharmaceutical composition comprising sodium picosulfate, magnesium oxide, citric acid, an alkaline agent and one or more pharmaceutically acceptable excipients, wherein sodium picosulfate is coated on a neutral carrier.

In another general aspect there is provided a stable pharmaceutical composition comprising sodium picosulfate, magnesium oxide, citric acid and one or more pharmaceutical excipients and retains at least 80% of the potency of active ingredients in the pharmaceutical composition after storage at 40° C. and 75% relative humidity for three months.

In another general aspect there is provided a process of preparing pharmaceutical composition comprising sodium picosulfate, magnesium oxide, citric acid, wherein the process comprises preparing granules of sodium picosulfate and a neutral carrier; drying the granules; mixing sodium picosulfate granules with citric acid, magnesium oxide, an alkaline agent and optionally, saccharin sodium and/or flavoring agent to prepare the final composition.

In another general aspect there is provided a method of bowel cleansing prior to X-ray examination, endoscopy or surgery comprising administering to said subject a pharmaceutical composition comprising sodium picosulfate coated on a neutral carrier, magnesium oxide, citric acid and one or more pharmaceutically acceptable excipients.

The details of one or more embodiments of the present invention are set forth in the description below. Other features, objects and advantages of the invention will be apparent from the description.

### DETAILED DESCRIPTION OF THE INVENTION

We have surprisingly found that compositions of sodium picosulfate, magnesium oxide and citric acid can be prepared using a neutral carrier and such compositions provide an improved homogeneous product with marked reduction in processing time.

The inventors have noticed that by judicial selection of excipients in its optimum concentrations, and particularly using a neutral carrier, the stable compositions having good physicochemical properties can be prepared.

Moreover, such formulations are also stable and may retain at least 80% of the potency of sodium picosulfate, magnesium oxide and citric acid in the pharmaceutical composition after storage at 40° C. and 75% relative humidity for three months.

The phrase “pharmaceutically acceptable” is employed herein to refer to those compounds, materials, compositions; and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The dosage form for oral delivery is in the form of granules. Herein the term granule(s) includes loose particles (such as particles which might collectively be termed a powder, including loose particles in the form of a powder which is known in the art as “powder for oral administration”).

As used herein, the term “an alkaline agent” includes but is not limited to one or more of sodium bicarbonate, potassium bicarbonate, calcium carbonate, sodium carbonate and lithium carbonate.

As used herein, the term “a neutral carrier” includes but is not limited to one or more of sugars, sugar alcohols and cellulose derivatives. Suitable sugars include, but are not limited to, sucrose, glucose, lactose, maltose, xylose, dextrose, fructose. Suitable sugar alcohols include, but are not limited to, sorbitol, mannitol, xylitol, maltitol, erythritol, isomalt, lactitol. Suitable cellulose derivatives include, but are not limited to, microcrystalline cellulose, powdered cellulose.

Embodiments of the present invention relate to pharmaceutical compositions of sodium picosulfate, magnesium oxide, citric acid, a neutral carrier and one or more pharmaceutically acceptable excipients.

The pharmaceutical composition of the present invention refers to pharmaceutical compositions which can be formulated into powder, granules, fine granules/micro-granules, pellets, wafers, sachets, tablets or capsules.

In one embodiment, the pharmaceutical composition comprising sodium picosulfate, magnesium oxide, citric acid, a neutral carrier and one or more pharmaceutically acceptable excipients.

In another embodiment, a homogeneous pharmaceutical composition comprising sodium picosulfate, magnesium oxide, citric acid, a neutral carrier and one or more pharmaceutical excipients.

In another embodiment, a pharmaceutical composition comprising sodium picosulfate, magnesium oxide, citric acid, a neutral carrier, an alkaline agent and one or more pharmaceutically acceptable excipients.

In another embodiment, a pharmaceutical composition comprising sodium picosulfate, magnesium oxide, citric acid, a neutral carrier and one or more pharmaceutically acceptable excipients, wherein sodium picosulfate is coated on a neutral carrier.

In another embodiment, a pharmaceutical composition comprising sodium picosulfate, magnesium oxide, citric acid, neutral carrier, an alkaline agent and one or more pharmaceutically acceptable excipients, wherein sodium picosulfate is coated on a neutral carrier.

In another embodiment, a pharmaceutical composition comprising sodium picosulfate, magnesium oxide, citric acid, lactose, potassium bicarbonate and one or more pharmaceutically acceptable excipients, wherein sodium picosulfate is coated on lactose.

In another embodiment, a stable pharmaceutical composition comprises sodium picosulfate, magnesium oxide, citric acid and one or more pharmaceutical excipients and retains at least 80% of the potency of active ingredients in the pharmaceutical composition after storage at 40° C. and 75% relative humidity for three months.

The pharmaceutically acceptable excipients for use in the pharmaceutical composition of sodium picosulfate, magnesium oxide and citric acid may include one or more diluents/fillers/bulking agents, binder, disintegrants, sweeteners/taste masking agents, colorants and flavors.

Suitable diluents/fillers/bulking agents include, but are not limited to, microcrystalline cellulose, di- or tri-basic calcium phosphate, crystalline cellulose, powdered cellulose, calcium carbonate, calcium sulphate, magnesium silicate, magnesium trisilicate, magnesium aluminium metasilicate (Neusilin), kaolin, starch, starch derivatives, magnesium carbonate, magnesium oxide and sugars such as sucrose, maltose, dextrose, lactose and the like.

Suitable binders include, but are not limited to, hydroxyethyl cellulose, hydroxypropyl cellulose, hydroxypropyl methylcellulose, carbomers, dextrin, ethyl cellulose, methylcellulose, shellac, zein, gelatin, gum arabic, polymethacrylates, polyvinyl pyrrolidone, polyvinyl alcohol, polyethylene glycol, carrageenan, polyethylene oxide, waxes, pullulan, agar, tragacanth, veegum, pregelatinized starch, sodium alginate, gums, sugars such as sucrose, maltose, dextrose, lactose, amylase, synthetic resins and the like.

Suitable disintegrants include, but are not limited to, Veegum (highly refined isomorphous silicate), crospovidone, cellulose, kaolin, crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof, Preferred disintegrants among these disintegrants include crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof. The amount of disintegrant in the pharmaceutical composition ranges from about 0.5% to about 10% by total weight of the composition.

Suitable taste masking agents include, but are not limited to, one or more of polymers, surfactants, sweeteners and flavors. Examples of polymers include one or more of cellulose acetate, polymethacrylates, cellulose derivatives such as hydroxypropylmethyl cellulose, hydroxypropyl cellulose, hydroxylethyl cellulose; and the like.

Suitable sweeteners include, but are not limited to, saccharides such as aspartame, sugar derivatives. Other examples of sweeteners comprise sodium saccharin; sugarless sweeteners, hydrogenated starch hydrolysates, alone or in combination.

Suitable flavors include, but are not limited to, cinnamon, wintergreen, eucalyptus, spearmint, peppermint, menthol, anise as well as fruit flavors such as apple, pear, peach, vanilla, strawberry, cherry, apricot, orange, watermelon, banana and the like; bean-derived flavors, such as coffee, cocoa and the like or mixtures thereof.

In another embodiment, there is provided granule or granules comprising a layer of sodium picosulfate coated on a neutral carrier. The granules may have a particle size (diameter) distribution wherein more than 85% of the granules have diameter between about 100 and about 900 μm.

In another embodiment, the pharmaceutical composition of sodium picosulfate, magnesium oxide and citric acid may be prepared by;
i) preparing granules of sodium picosulfate and a neutral carrier;
ii) drying the granules;
iii) mixing citric acid, magnesium oxide, an alkaline agent and optionally, a sweetener and/or a flavoring agent; and mixing the granules of step ii) with the mixture of step iii) to prepare the final composition.

In another embodiment, granules of sodium picosulfate and a neutral carrier may be prepared by:
i) preparing wet mass of sodium picosulfate and a neutral carrier;
ii) extruding the wet mass;
iii) spheronizing the extrudes to obtain spheres;
iv) drying the spheres; and
v) screening the dried spheres to obtain the granules.

In another embodiment, granules of sodium picosulfate and a neutral carrier may be prepared by spraying solution of sodium picosulfate on a neutral carrier.

In another embodiment, granules of sodium picosulfate and a neutral carrier may be prepared by:
i) passing sodium picosulfate and the neutral carrier through the mesh;
ii) loading the mixture on the roller compactor to get slugs; and
iii) milling these slugs and passing through co-mill to get properly sized granules.

The invention further provides a method of bowel cleansing prior to X-ray examination, endoscopy or surgery in a patient comprising administering to said subject a pharmaceutical composition comprising sodium picosulfate, magnesium oxide and citric acid and one or more pharmaceutical excipients.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLE 1

| | | Quantity |
|---|---|---|
| Sr. No. | Ingredient | (% w/w) |
| 1. | Sodium Picosulfate | 0.06 |
| 2. | Magnesium oxide | 20 |
| 3. | Citric acid | 68.57 |
| 4. | Potassium bicarbonate | 2.4 |
| 5. | Lactose | 8.11 |
| 6. | Saccharin sodium | 0.57 |
| 7. | Orange flavor | 0.29 |

### Process:

Granules of lactose and sodium picosulfate were prepared by spraying aqueous solution of sodium picosulfate on powder bed of lactose. Granules were then dried and admixed with some quantity of lactose, saccharin sodium and orange flavor. This mixture was then mixed with a mixture of citric acid, magnesium oxide and potassium bicarbonate to prepare the final composition. The final composition was packed in sachets.

### EXAMPLE 2

| Sr. No. | Ingredient Quantity | (% w/w) |
|---|---|---|
| 1. | Sodium Picosulfate | 0.06 |
| 2. | Magnesium oxide | 20 |
| 3. | Citric acid | 68.57 |
| 4. | Sodium bicarbonate | 2.4 |
| 5. | Mannitol | 8.11 |
| 6. | Saccharin sodium | 0.57 |
| 7. | Orange flavor | 0.29 |

### Process:

Granules of mannitol and sodium picosulfate were prepared by spraying aqueous solution of sodium picosulfate on powder bed of mannitol. Granules were then dried and admixed with some quantity of mannitol, saccharin sodium and orange flavor. This mixture was then mixed with a mixture of citric acid, magnesium oxide and sodium bicarbonate to prepare the final composition. The final composition was packed in sachets.

We claim,

1. A pharmaceutical composition comprising:
a) granules of sodium picosulfate and a neutral carrier, and
b) a mixture comprising citric acid, magnesium oxide, an alkaline agent and one or more pharmaceutically acceptable excipients,

2. The pharmaceutical composition according to claim 1, wherein the neutral carrier comprising one or more of saccharides, sugar alcohols and cellulose derivatives.

3. The pharmaceutical composition according to claim 1, wherein the neutral carrier is lactose.

4. The pharmaceutical composition according to claim 1, wherein the alkaline is agent comprising one or more of sodium bicarbonate, potassium bicarbonate, calcium carbonate, sodium carbonate and lithium carbonate.

5. The pharmaceutical composition according to claim 1, wherein the composition is in the form of capsules or sachets.

6. The pharmaceutical composition according to claim 1 retains at least 80% of the potency of active ingredients in the pharmaceutical composition after storage at 40° C. and 75% relative humidity for three months.

7. A process for preparing a pharmaceutical composition comprising:
i) preparing granules of sodium picosulfate and a neutral carrier;
ii) drying the granules;
iii) mixing citric acid, magnesium oxide, an alkaline agent and one or more pharmaceutically acceptable excipient; and
iv) mixing the granules of step ii) with the mixture of step iii) to prepare the final composition.

8. The process according to claim 7, wherein the granules are prepared by applying solution or suspension of sodium picosulfate onto the neutral carrier.

9. The process according to claim 7, wherein the granules are prepared by:
i) preparing wet mass of sodium picosulfate and the neutral carrier;
ii) extruding the wet mass;
iii) spheronizing the extrudes to obtain spheres;
iv) drying the spheres; and
v) screening the dried spheres to obtain the granules.

10. The process according to claim 7, wherein the granules are prepared by;
i) passing sodium picosulfate and the neutral carrier through the mesh;
ii) loading the mixture on roller compactor to get slugs; and
iii) milling these slugs and passing through co-mill to get properly sized granules.

11. The composition according to claim 1, wherein more than 85% of the granules have a diameter between about 100 μm and about 900 μm.

12. A pharmaceutical composition comprising:
a) granules of sodium picosulfate and lactose; and
b) a mixture comprising citric acid, magnesium oxide, potassium bicarbonate and one or more pharmaceutically acceptable excipients, wherein sodium picosulfate is coated onto lactose.

13. A method of bowel cleansing prior to X-ray examination, endoscopy or surgery in a patient comprising administering to a human patient in need thereof the pharmaceutical composition according to claim 1.
